(19) United States
(12) Patent Application Publication
(10) Pub. No.: US 2023/0329963 A1
(43) Pub. Date: Oct. 19, 2023

(54) Vibrating Silicone Dilator

(71) Applicant: Nicole Williams, Douglasville, TN (US)

(72) Inventor: Nicole Williams, Douglasville, TN (US)

(21) Appl. No.: 18/296,708

(22) Filed: Apr. 6, 2023

Related U.S. Application Data
(60) Provisional application No. 63/362,942, filed on Apr. 13, 2022.

(51) Int. Cl. A61H 19/00; A61H 23/02

(52) U.S. Cl. CPC A61H 19/44; A61H 23/02; A61H 2201/1692; A61H 2201/0153

(57) ABSTRACT

Various implementations include a dilator including a housing, a coating layer, and a sleeve. The housing includes a handle body and a protrusion. The protrusion has a first protrusion end coupled to a second handle end, a second protrusion end, and a protrusion outer surface. A first end of the coating layer defines a coating opening extending toward a second end of the coating layer and defining a coating inner surface. The second protrusion end is disposed within the coating opening to couple the coating layer to the housing. A first end of the sleeve defines a sleeve opening extending toward a second end of the sleeve and defining a sleeve inner surface. The second end of the coating layer is removably disposable within the sleeve opening to couple the sleeve to the coating layer and the housing.

### FIELD

This disclosure relates to the field of health and wellness. More particularly, this disclosure relates to a dilator for exercising and training of the pelvic floor and vaginal muscles.

### BACKGROUND

Dilators may be used for strengthening muscles or for other wellness purposes. Although some dilators exist, typically a user must purchase different sized dilators. Existing dilators are also typically formed of a hard material, such as a hardened plastic, which may increase discomfort during use of the dilator. Other existing dilators may not include features such as vibrating features, which may enhance use of or otherwise increase enjoyment of use of the dilator.

### SUMMARY

Various implementations include a dilator including a housing, a coating layer, and a sleeve. The housing includes a handle body and a protrusion. The handle body has a first handle end and a second handle end opposite and spaced apart from the first handle end. The protrusion has a first protrusion end coupled to the second handle end, a second protrusion end opposite and spaced apart from the first protrusion end, and a protrusion outer surface extending from the first protrusion end to the second protrusion end. The coating layer has a first coating end and a second coating end opposite and spaced apart from the first coating end. The first coating end defines a coating opening extending toward the second coating end. The coating layer has a coating inner surface defined by the coating opening and a coating outer surface spaced radially outwardly from the coating inner surface. The second protrusion end is disposed within the coating opening to couple the coating layer to the housing. The sleeve has a first sleeve end and a second sleeve end opposite and spaced apart from the first sleeve end. The first sleeve end defines a sleeve opening extending toward the second sleeve end. The sleeve has a sleeve inner surface defined by the sleeve opening and a sleeve outer surface spaced radially outwardly from the sleeve inner surface. The second coating end is removably disposable within the sleeve opening to couple the sleeve to the coating layer and the housing.

In some implementations, the dilator further includes a vibrator motor disposed within the housing. In some implementations, the vibrator motor is configured to cause vibration of a sleeve coupled to the coating layer. In some implementations, the vibrator motor is disposed within the protrusion of the housing. In some implementations, the dilator further includes a battery disposed within the housing. In some implementations, the battery is in electrical communication with the vibrator motor.

In some implementations, the sleeve includes silicone. In some implementations, the coating layer includes silicone. In some implementations, the sleeve and the coating layer include a same material.

In some implementations, one of the housing outer surface and the coating inner surface defines a circumferentially extending first groove, and an other of the coating inner surface and the housing outer surface includes a circumferentially extending first protrusion sized to be disposed within the first groove. In some implementations, the housing outer surface defines the first groove, and the coating inner surface includes the first protrusion.

In some implementations, one of the housing outer surface and the sleeve inner surface defines a circumferentially extending second groove, and an other of the sleeve inner surface and the housing outer surface includes a circumferentially extending second protrusion sized to be disposed within the second groove. In some implementations, the housing outer surface defines the second groove, and the sleeve inner surface includes the second protrusion.

In some implementations, the coating opening is a same size as the protrusion outer surface such that the entire protrusion outer surface contacts the coating inner surface.

In some implementations, the sleeve opening is a same size as the coating outer surface such that the entire coating outer surface contacts the sleeve inner surface when the second coating end is disposed within the sleeve opening to couple the sleeve to the coating layer.

In some implementations, the sleeve is a first sleeve and the dilator further includes a second sleeve having at least one larger outer dimension than the first sleeve. In some implementations, the second coating end is removably disposable within the sleeve opening of the second sleeve to couple the second sleeve to the coating layer. In some implementations, the outer dimension is a length as measured from the first sleeve end to the second sleeve end of the respective sleeve. In some implementations, the length is in the range of 10 cm-18 cm. in some implementations, the outer dimension is a largest diameter as measured circumferentially around a portion of the sleeve adjacent the first sleeve end of the respective sleeve. In some implementations, the largest diameter is in the range of 20 mm-35 mm.

In some implementations, the sleeve outer surface defines a textured surface. In some implementations, the textured surface includes a plurality of bumps.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows a cross-sectional side view of a dilator according to one embodiment of the present disclosure;

FIGS. 2A-2D show cross-sectional side views of varying sized sleeves fitted over an end of a dilator according to one embodiment of the present disclosure;

FIGS. 3 and 4 show cross sectional views of a dilator according to one embodiment of the present disclosure; and

FIGS. 5A-5E show views of a dilator having a sleeve located thereon according to one embodiment of the present disclosure.

FIG. 6A shows a view of a dilator having a sleeve located thereon according to one embodiment of the present disclosure.

FIGS. 6B-6E show views of sleeve for a dilator according to embodiments of the present disclosure.

### DETAILED DESCRIPTION

Various terms used herein are intended to have particular meanings. Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

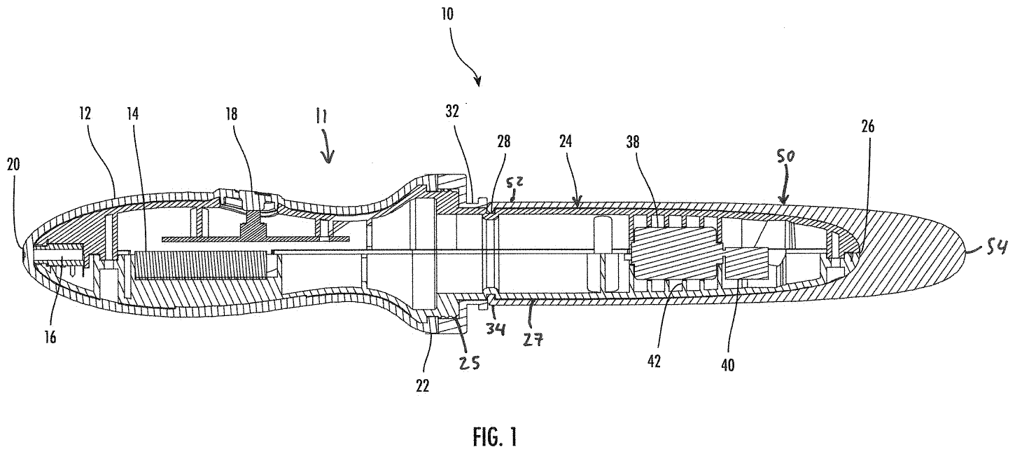

FIG. 1 shows a basic embodiment of a dilator 10. The dilator 10 includes a soft pliable section, such as a section formed of silicone, to provide increased comfort for use of the dilator 10. Portions of the dilator 10 may be swappable such that a size of the dilator 10 may be adjusted. Further, the dilator 10 may include a vibrating feature to enhance use of the dilator 10.

The dilator 10 includes a housing 11, a coating layer 50, and a sleeve 70. The housing 11 includes a handle body 12 located towards a first end of the dilator 10 and a protrusion 24 coupled to the handle body 12.

The handle body 12 has a first handle end 20 and a second handle end 22 opposite and spaced apart from the first handle end 20. The handle body 12 may be hollow and shaped to contain components of the dilator 10 therein. The handle body 12 may be shaped to be grasped in the hand of a user and may include shaped portions to allow comfortable holding in the hand of a user during use of the dilator 10. The handle body 12 may be formed of a harder material, such as plastic, and may include softer materials located on a surface thereof to increase a grip or comfort in holding the dilator 10.

A power source 14 may be located within the handle body 12. An example of a suitable power source 14 may include, for example, a battery or a capacitor that is located within the handle body 12. The handle body 12 may further include a port 16 located through the handle body 12 for charging the power source 14. The handle body 12 may further include one or more buttons 18 located on an outer surface of the handle body 12 for activating the dilator 10 as described in greater detail below. The handle body 12 may be substantially watertight to prevent water from entering an interior of the handle body 12 and contacting components within the handle body 12, such as the power source 14.

The handle body 12 extends from the first handle end 20 to the second handle end 22 opposite and spaced apart from the first handle end 20. The second handle end 22 of the handle body 12 may be flared or otherwise be formed having a diameter that is greater than a portion of the handle body 12 that is gripped by a user.

The protrusion 24 has a first protrusion end 25 coupled to the second handle end 22 and a second protrusion end 26 opposite and spaced apart from the first protrusion end 25. The protrusion 24 further has a protrusion outer surface 27 extending from the first protrusion end 25 to the second protrusion end 26. The protrusion 24 extends from the second handle end 22 of the handle body 12 to the second protrusion end 26 that is distal from the second handle end 22 of the handle body 12. The protrusion 24 may be formed of a harder material, such as plastic, and may be formed of a material that is similar to or the same as a material of the handle body 12. The protrusion 24 may be substantially hollow such that vibrating components of the dilator 10 may be located within the protrusion 24.

The protrusion 24 may be shaped such that a diameter of the protrusion 24 is less than a diameter of the handle body 12 at the flared second handle end 22 of the handle body 12. The protrusion 24 may taper in width such that the protrusion 24 is narrower at the second protrusion end 26 of the protrusion 24 relative to a diameter of the protrusion 24 at the flared second handle end 22 of the handle body 12. The protrusion 24 may further include a circumferentially extending first groove 28 defined by the protrusion 24 adjacent or proximate to the flared second handle end 22 of the handle body 12. The first groove 28 may circumscribe the protrusion 24 towards the flared second handle end 22 of the handle body 12.

The coating layer 50 may be formed over the protrusion 24. The coating layer 50 has a first coating end 52 and a second coating end 54 opposite and spaced apart from the first coating end 52. The first coating end 52 defines a coating opening 60 extending toward the second coating end 54. The coating layer 50 has a coating inner surface 62 defined by the coating opening 60 and a coating outer surface 64 spaced radially outwardly from the coating inner surface 62.

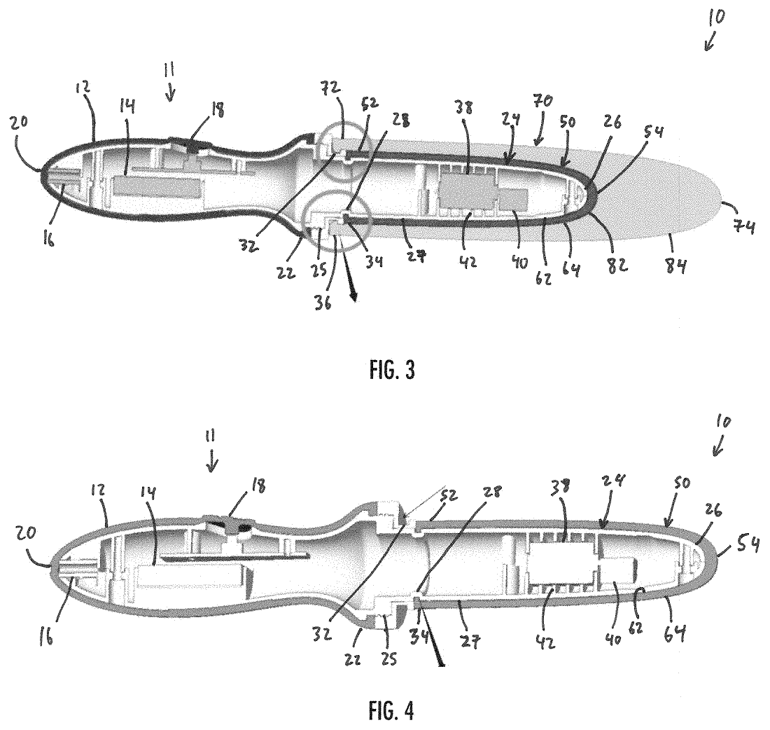

As shown in FIGS. 3 and 4, the second protrusion end 26 of the protrusion 24 is disposed within the coating opening 60 to couple the coating layer 50 to the housing 11. The coating layer 50 may be formed directly on the protrusion 24. The coating inner surface 62 may include a radially inwardly extending first flange 34 shaped to engage the first groove 28 of the protrusion 24 to secure the coating layer 50 thereon. However, in some implementations, the housing outer surface includes the first flange, and the coating inner surface defines a circumferentially extending first groove that is engageable by the first flange of the housing.

The coating inner surface 62 of the coating layer 50 may have the same shape and size as the protrusion outer surface 27 such that the entire coating inner surface 62 of the coating layer 50 is in contact with substantially all of the protrusion outer surface 27 of the protrusion 24 as shown in FIG. 1. The outer surface of the coating layer 50 may have a diameter that is greater than a diameter of the protrusion 24 and may be shaped similar to an outer shape of the protrusion 24.

The housing 11 further includes a circumferentially extending second groove 32 that is defined by the second handle end 22 or the first protrusion end 25, as shown in FIGS. 3 and 4. The second groove 32 may have a diameter that is greater than a diameter of the first groove 28. The second groove 32 may be formed adjacent to the flared second handle end 22 of the handle body 12 and is shaped to receive a variety of sizes of sleeves 70 over the coating layer 50 and the protrusion 24 of the dilator 10 as discussed in greater detail below.

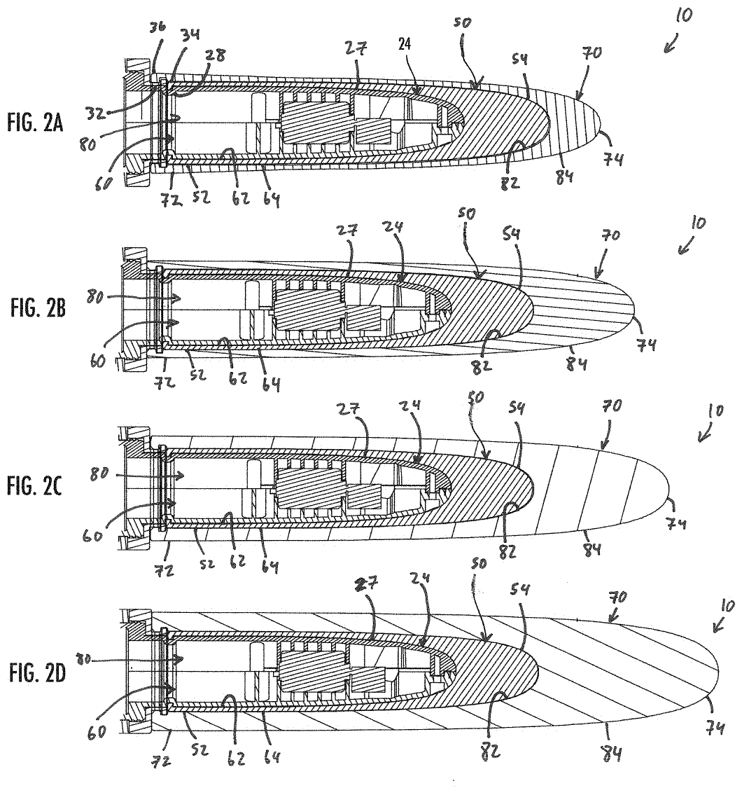

Referring to FIGS. 2A-2D, a sleeve 70 may be shaped to fit over the coating layer 50 and the protrusion 24 of the housing 11 of the dilator 10. The sleeve 70 has a first sleeve end 72 and a second sleeve end 74 opposite and spaced apart from the first sleeve end 72. The first sleeve end 72 defines a sleeve opening 80 extending toward the second sleeve end 74. The sleeve 70 has a sleeve inner surface 82 defined by the sleeve opening 80 and a sleeve outer surface 84 spaced radially outwardly from the sleeve inner surface 82. The second coating end 54 is removably disposable within the sleeve opening 80 to couple the sleeve 70 to the coating layer 50 and the housing 11.

The sleeve 70 may be formed of a silicone material, like the coating layer 50, or can otherwise be formed of the same material as the coating layer 50. The sleeve inner surface 82 of the sleeve opening 80 may have the same shape and size as the coating outer surface 64 such that the coating outer surface 64 fit closely against the sleeve inner surface 82 when the second coating end 54 is disposed within the sleeve opening 80 to couple the sleeve 70 to the coating layer 50. The sleeve 70 may include a circumferentially extending second flange 36 that is shaped to fit within the second groove 32 to secure the sleeve 70 on the protrusion 24 and coating layer 50 of the dilator 10. However, in some implementations, the housing outer surface includes the second flange, and the sleeve inner surface defines a circumferentially extending second groove that is engageable by the second flange of the housing.

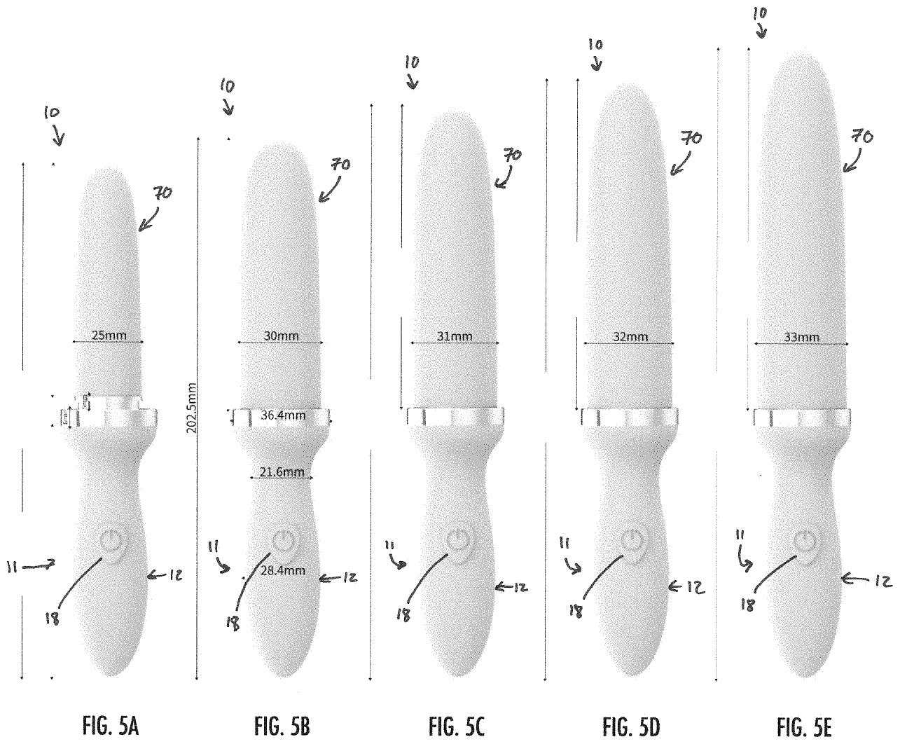

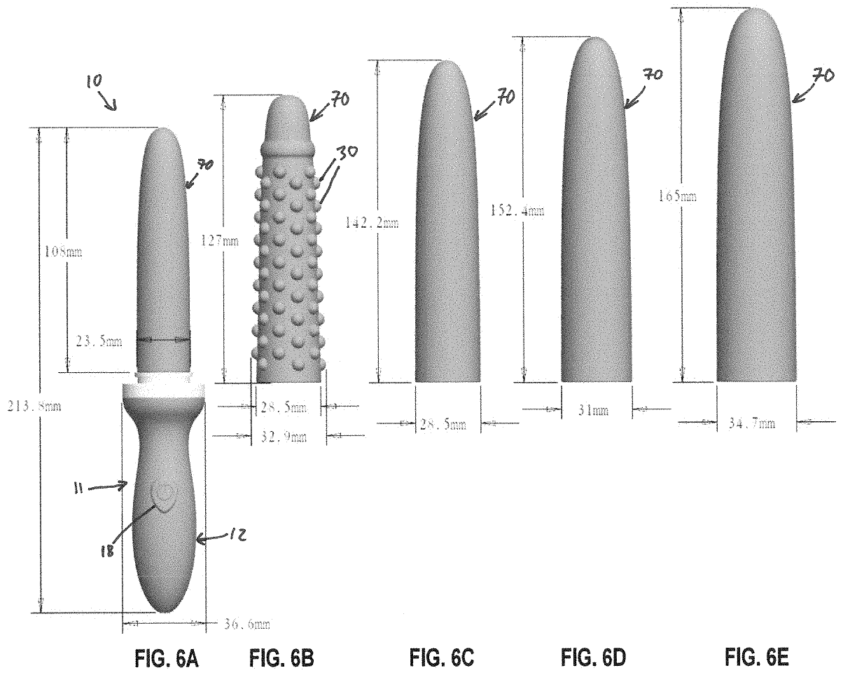

The sleeve 70 may vary in size and may be swappable over the protrusion 24 of the dilator 10. For example, the dilator 10 may include a first sleeve 70 and a second sleeve 70. The second sleeve 70 may have at least one larger outer dimension, such as a length or a diameter, than the first sleeve 70. However, the first sleeve 70 and the second sleeve 70 have the same shape and size sleeve openings 80 such that each of the first sleeve 70 and the second sleeve 70 are removably couplable to the coating layer 50 and the protrusion 24 of the dilator 10. In some implementations, the dilator may include any number of removable sleeves, such as those shown in FIGS. 5A-6E. The various sleeves 70 may vary in size, such as having a length of from about 10 cm to about 18 cm. Further the sleeve 70 may vary in width, having a width that varies from about 20 mm to about 35 mm. In some implementations, one or more sleeves may include a sleeve outer surface that defines a textured surface, such as the plurality of bumps shown in FIG. 6B.

Referring again to FIG. 1, the dilator 10 may be configured to vibrate. The dilator 10 may include a vibrator motor 38. The vibrator motor 38 may be located within the protrusion 24 of the dilator 10, such as towards the second protrusion end 26 of the protrusion 24. However, in some implementations, the vibrator motor can be located in any portion of the housing. The vibrator motor 38 may include an offset weight 40 that is driven by the vibrator motor 38 to induce vibrations in the dilator 10. The vibrator motor 38 may be in electrical communication with the power source 14 to power the vibrator motor 38.

The vibrator motor 38 may be supported within the protrusion 24 on a plurality of support members 42. The plurality of support members 42 may circumscribe the vibrator motor 38 and mechanically couple the vibrator motor 38 with the protrusion 24 to transfer vibrations from the vibrator motor 38 to the protrusion 24 and subsequently to the coating layer 50 and sleeve 70 mounted thereon.

The dilator 10 advantageously allows for various sizes of the sleeves 70 to be removably mounted over the protrusion 24 and coating layer 50 of the dilator 10. The sleeves 70 may be readily swapped by disengaging the sleeve 70 from the second groove 32 and sliding a different size sleeve 70 over the protrusion 24 of the dilator 10. Further, the arrangement of the sleeve 70 in relation to the coating layer 50 and the protrusion 24 enhances vibrations generated by the vibrator motor 38 such that vibrations may be felt through the sleeve 70.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A dilator comprising:
a housing comprising:
a handle body having a first handle end and a second handle end opposite and spaced apart from the first handle end, and
a protrusion having a first protrusion end coupled to the second handle end, a second protrusion end opposite and spaced apart from the first protrusion end, and a protrusion outer surface extending from the first protrusion end to the second protrusion end;
a coating layer having a first coating end and a second coating end opposite and spaced apart from the first coating end, wherein the first coating end defines a coating opening extending toward the second coating end, wherein the coating layer has a coating inner surface defined by the coating opening and a coating outer surface spaced radially outwardly from the coating inner surface, wherein the second protrusion end is disposed within the coating opening to couple the coating layer to the housing; and
a sleeve having a first sleeve end and a second sleeve end opposite and spaced apart from the first sleeve end, wherein the first sleeve end defines a sleeve opening extending toward the second sleeve end, wherein the sleeve has a sleeve inner surface defined by the sleeve opening and a sleeve outer surface spaced radially outwardly from the sleeve inner surface, wherein the second coating end is removably disposable within the sleeve opening to couple the sleeve to the coating layer and the housing.

2. The dilator of claim 1, further comprising a vibrator motor disposed within the housing, wherein the vibrator motor is configured to cause vibration of a sleeve coupled to the coating layer.

3. The dilator of claim 2, wherein the vibrator motor is disposed within the protrusion of the housing.

4. The dilator of claim 2, further comprising a battery disposed within the housing, wherein the battery is in electrical communication with the vibrator motor.

5. The dilator of claim 1, wherein the sleeve comprises silicone.

6. The dilator of claim 1, wherein the coating layer comprises silicone.

7. The dilator of claim 1, wherein the sleeve and the coating layer comprise a same material.

8. The dilator of claim 1, wherein one of the housing outer surface and the coating inner surface defines a circumferentially extending first groove, and an other of the coating inner surface and the housing outer surface includes a circumferentially extending first flange sized to be disposed within the first groove.

9. The dilator of claim 8, wherein the housing outer surface defines the first groove, and the coating inner surface includes the first flange.

10. The dilator of claim 1, wherein one of the housing outer surface and the sleeve inner surface defines a circumferentially extending second groove, and an other of the sleeve inner surface and the housing outer surface includes a circumferentially extending second flange sized to be disposed within the second groove.

11. The dilator of claim 10, wherein the housing outer surface defines the second groove, and the sleeve inner surface includes the second flange.

12. The dilator of claim 1, wherein the coating opening is a same size as the protrusion outer surface such that the entire protrusion outer surface contacts the coating inner surface.

13. The dilator of claim 1, wherein the sleeve opening is a same size as the coating outer surface such that the entire coating outer surface contacts the sleeve inner surface when the second coating end is disposed within the sleeve opening to couple the sleeve to the coating layer.

14. The dilator of claim 1, wherein the sleeve is a first sleeve, further comprising a second sleeve having at least one larger outer dimension than the first sleeve, wherein the second coating end is removably disposable within the sleeve opening of the second sleeve to couple the second sleeve to the coating layer.

15. The dilator of claim 14, wherein the outer dimension is a length as measured from the first sleeve end to the second sleeve end of the respective sleeve.

16. The dilator of claim 15, wherein the length is in the range of 10 cm-18 cm.

17. The dilator of claim 14, wherein the outer dimension is a largest diameter as measured circumferentially around a portion of the sleeve adjacent the first sleeve end of the respective sleeve.

18. The dilator of claim 17, wherein the largest diameter is in the range of 20 mm-35 mm.

19. The dilator of claim 1, wherein the sleeve outer surface defines a textured surface.

20. The dilator of claim 19, wherein the textured surface comprises a plurality of bumps.
